(19) United States
(12) Patent Application Publication
(10) Pub. No.: US 2026/0273337 A1
(43) Pub. Date: Sep. 17, 2026

(54) INTELLIGENT ADAPTOR FOR A BREATHING TRAINING DEVICE

(71) Applicant: Powerbreathe Holdings Limited, Stratford-Upon-Avon Warwickshire (GB)

(72) Inventors: Harjinder Brar, Stratford-Upon-Avon Warwickshire (GB); Darren Lam, Stratford-Upon-Avon Warwickshire (GB); Omar Muttawa, Stratford-Upon-Avon Warwickshire (GB)

(73) Assignee: Powerbreathe Holdings Limited, Pembridge, Leominster Herefordshire (GB)

(21) Appl. No.: 19/469,420

(22) PCT Filed: Mar. 11, 2024
(86) PCT No.: PCT/EP2024/056435

§ 371 Date: Sep. 26, 2025

(30) Foreign Application Priority Data
Mar. 30, 2023 (GB) 2304692.3
Sep. 8, 2023 (GB) 2313709.4

(51) Int. Cl. A63B 23/18

(52) U.S. Cl. CPC A63B 23/18; A63B 2220/56; A63B 2225/50

(57) ABSTRACT

An intelligent adaptor 10 for a breathing training device comprising a first end 12 to attach to a mouthpiece of the device, and a second end 14 to be received into a user's mouth. The ends 12, 14 are connected by a passageway 16 that provides a sealed chamber between the user's lungs and the mouthpiece, such that if the measured pressure within the passageway 16 crosses a threshold value, a resistance valve within the device moves from a first to a second configuration to allow the user to breathe freely through the device. The adaptor 10 includes a control system 20 comprising a pressure sensor 22 to measure user-generated pressure within the passageway 16 during a training session; and, a transceiver 24 to receive this pressure measurement data and transmit the data to a computing device for analysis.

### FIELD OF INVENTION

The present invention relates to an intelligent adaptor for a breathing training device, and systems and methods relating to the same, as well as a computer system for monitoring the breathing performance of a user when using such an adaptor.

### BACKGROUND TO THE INVENTION

Breathing training devices are typically used for training the muscles that control breathing, with the goal of improving respiratory muscle strength and endurance. They work by providing resistance to a user's inspiratory (breathing in) efforts or expiratory (breathing out) efforts. The user typically inhales or exhales through the device against a resistance, such as a spring-loaded valve or a magnetic resistance, which helps to strengthen the muscles used for breathing.

Such devices can be beneficial for people with chronic respiratory conditions, such as asthma, chronic obstructive pulmonary disease (COPD), and bronchitis, as well as for athletes and individuals looking to improve their respiratory fitness.

The present invention seeks to provide means to improve upon such breathing training devices.

### SUMMARY OF THE INVENTION

In accordance with a first aspect of the invention, there is provided an intelligent adaptor for a breathing training device, the adaptor comprising:
a first end configured to attach to a mouthpiece of a breathing training device;
a second end configured to be received into a mouth of a user, such that a seal is created in use around the second end of the adaptor;
the first and second ends being connected by a passageway, the passageway being configured to provide a sealed chamber, in use, between at least the user's lungs and the mouthpiece of the breathing training device, such that if a measured pressure within the passageway crosses a threshold value, a resistance valve located within the breathing training device moves from a first to a second configuration to allow the user to breathe freely through the device; and,
a control system comprising:
   a pressure sensor configured to measure a user-generated pressure within the passageway at pre-defined increments of time during a training session; and,
   a transceiver configured to receive pressure measurement data from the pressure sensor, and transmit the data to a computing device for analysis.

The present invention recognises that most mechanical breathing training devices, or lung muscle trainers, require the user to set the required resistance level by manually turning a dial on the device to adjust the resistance load provided by the valve. However, such devices do not allow a user to monitor their performance during a training session and then receive detailed feedback on a number of breathing performance indicators, nor do they allow for monitoring their progress over several training sessions.

The present invention therefore provides an intelligent adaptor that can be retrofitted to the mouthpiece of current breathing training devices, to form a new mouthpiece with added functionality. The intelligent adaptor communicates with a computing device to provide the user with feedback on their training sessions. Prior to commencing a training session, the user can enter their target resistance level and intended activity into the computing device. Then during and after the training session, the computing device provides feedback to the user, such as how close they were to reaching their target resistance level, or if they succeeded in reaching their target resistance level. Such functionality allows a user to monitor their performance throughout their training session, and make any necessary adjustments. It can also allow the user to track their progress over several (or all) training sessions. As such, an advantage of aspects and embodiments of the present invention is that the measured and calculated values relating to the usage of the training device are displayed to the user in real-time, to allow the user or their trained medical professional (such as a physiotherapist) to monitor, track, and optimise the usage of their mechanical training device, which was previously not capable of providing such information and feedback.

Moreover, the computing device can analyse the measured and calculated values to provide the user with automated suggestions on how to improve their usage of the device and optimise their training. In this manner, the computing device can act as a virtual coach on the correct usage of the training device.

The pressure sensor may be configured to measure the pressure generated by the user's lungs at the location of the sensor at the pre-defined increments of time. The pre-defined increments of time may be at least 42 ms, optionally from 42 ms to 210 ms, optionally from 100 ms to 200 ms, optionally at most 210 ms. As such, the sampling rate can be up to around 24 Hz. The pre-defined increments of time may be chosen such that the pressure sensor can adequately detect when a user begins an inhalation, ends an inhalation, begins an exhalation, and ends an exhalation.

The user can generate pressure in the passageway in use during an inhalation or an exhalation.

The transceiver may be configured to communicate with the computing device via a wired or wireless connection. In particular, the transceiver can be configured to communicate with the computing device via a Bluetooth connection. The computing device may be a mobile device comprising Bluetooth functionality, such as a smartphone or laptop.

The computing device may comprise a software application, the software application being configured to:
receive input from a user relating to a target resistance level provided by the resistance valve; thereby determining the threshold value which must be crossed during a training session to move the valve from the first to the second configurations;
receive pressure measurement data from the pressure sensor; and,
determine at least one breathing performance indicator of the user based at least on a comparison of the target resistance level with the pressure measurement data from the pressure sensor.

In embodiments, the at least one breathing performance indicator of the user may additionally be based on one or more of:
the number of times the pressure measurement data from the pressure sensor fell within or were greater than the target resistance level for more than a pre-defined amount of time (successful attempts);
the number of times the actual pressure measurement data fell within or were greater than the target resistance level for less than a pre-defined amount of time (unsuccessful attempts);
the number of times the actual pressure measurement data was less than the target resistance level (unsuccessful attempts);
the amount of time taken to reach the threshold pressure required to move the valve from the first to the second configurations;
the inhalation or exhalation time of the previous successful attempt;
the maximum air pressure generated, and/or the average air pressure generated over the training session;
the inhalation time of the previous inhalation and/or the average inhalation time over the training session;
the exhalation time of the previous exhalation and/or the average exhalation time;
the average resistance level achieved during the training session;
the total measurement time, which defines the length of a training session; and,
the average pressure time product (PTP) achieved and/or the total pressure time product (PTP) achieved during the training session.

As such, an attempt may only be recorded as successful if the user 1) generates an air pressure falling within or above their target resistance level, and 2) remains within or above their target resistance level for a minimum amount of time. If either of these conditions are not met, the attempt may be registered as unsuccessful.

In embodiments, the software application may be further configured to display the pressure measurement data, for example, plotted against the time increment data, in real-time during a training session. The software application can additionally be configured to display in real-time the at least one breathing performance indicator of the user during a training session. The at least one breathing performance indicator may be updated and displayed in real-time as a training session progresses. In embodiments, the at least one breathing performance indicator can be compared to the same breathing performance indicator from a previous training session, such that the user can monitor their progress. An advantage of embodiments of the invention is that the user or a trained medical professional (such as a physiotherapist) can monitor, track, and optimise the usage of the training device to allow the user to achieve their training goals.

In some embodiments, two or more breathing performance indicators can be combined to form at least one breathing performance indicator which is more user-friendly. The software application can additionally be configured to update and display the at least one user-friendly breathing performance indicator in real-time during a training session. The at least one user-friendly breathing performance indicator can be more easily understood by a user, thereby making it easier to monitor their performance.

The software application may be configured to analyse the pressure measurement data and/or the at least one breathing performance indicator, for example by comparing to previous values and/or to suitable threshold values, and can provide the user with one or more automated suggestions on how to improve their usage of the device and optimise their training. In this manner, the computing device can act as a coach on the correct usage of the training device.

The intelligent adaptor may further comprise a power source, such as a rechargeable power source. For example, the intelligent adaptor can comprise a battery. The intelligent adaptor can comprise an input for connecting the adaptor to an external power supply. For example, the external power supply may be a mains power supply, and preferably may be used to recharge the battery via a power adaptor.

In embodiments, the adaptor may be further configured to receive a selectively removable plug. In embodiments, the passageway at the first end can be configured to at least partially receive the selectively removeable plug. In use, the passageway at the first end can attach to either a breathing training device and/or a plug. In some embodiments, in use, the plug can substantially occlude (i.e., substantially restrict or block or close) the passageway at the first end, thereby preventing (or at least minimising) air flow between the user's lungs and the plug, and enabling the user to generate pressure (e.g., an increase or decrease in pressure) within the passageway under isometric conditions.

Herein, “isometric conditions” are understood to mean a change in pressure for a constant volume of air. The pressure generated under isometric conditions is measurable by the pressure sensor. In such embodiments, the adaptor can be used to perform isometric respiratory muscle strength tests.

In accordance with a second aspect of the invention, there is provided a system for monitoring the breathing performance of a user, the system comprising:
a breathing training device; the breathing training device comprising a mouthpiece and a resistance valve, and arranged such that the resistance valve moves from a first to a second configuration if the user-generated air pressure within the device crosses a threshold value; and,
an intelligent adaptor according to the first aspect of the invention;
wherein the intelligent adaptor is mounted onto the mouthpiece of the breathing training device, and provides a passageway configured to provide a sealed chamber in use between at least the user's lungs and the mouthpiece of the breathing training device.

As such, the intelligent adaptor may be retrofitted to any suitable breathing training device. For example, the training devices developed by the Applicant, such as (but not limited to) the POWERbreathe Plus range, the POWERbreathe Medic Plus range, and the POWERbreathe EX1 range.

The first configuration can be a closed position, and the second configuration can be an open position. Alternatively, the first configuration can be an open position, and the second configuration can be a closed position.

The resistance valve may be configured to move from the first to the second configurations by one of an inhalation or an exhalation. As such, the breathing training device can either be an inspiratory training device or an expiratory training device.

The resistance valve can be an adjustable valve. The resistance load provided by the valve can be adjusted to increase or decrease the threshold value at which the valve moves from the first to the second configurations.

The resistance load provided by the resistance valve may be varied incrementally, such as in increments of 8 cmH₂O, 16 cmH₂O, or 25 cmH₂O, up to a maximum resistance load. Each increment can correspond to a resistance level, whereby each resistance level can span a range of resistance loads, for example from 8 cmH₂O-16 cmH₂O, 16 cmH₂O-24 cmH₂O, and so on. There may be up to 10 progressively increasing resistance levels, optionally more than 10, such as 11, progressively increasing resistance levels, each defined by a range of resistance loads. In use, the user can choose their target resistance level prior to beginning a training session.

Each resistance level can comprise a threshold value which must be crossed to move the valve from the first to the second configurations. The pre-defined increments of time can be sufficient to allow the pressure sensor to adequately detect when a user moves into and out of a resistance level.

The resistance valve can comprise an adjustable spring. In use, adjusting the physical properties of the spring (such as the length or amount of torsion) can change the threshold value, and therefore make it harder or easier to move the valve from the first to the second configurations.

In accordance with a third aspect of the invention, there is provided a method of monitoring the breathing performance of a user using the system according to the second aspect of the invention, the method comprising:
inputting a target resistance level into a computing device;
placing the second end of the intelligent adaptor into a user's mouth, such that a seal is created around the second end of the adaptor;
generating a pressure within the passageway between the user and the resistance valve;
measuring the air pressure within the passageway at pre-defined increments of time;
transmitting the air pressure measurements to a computing device;
determining at least one breathing performance indicator of the user based at least on a comparison of the target resistance level with the air pressure measurements; and
displaying the pressure measurement data and/or the at least one breathing performance indicator, on one or more display screens of the computing device for viewing by the user.

The method may further comprise analysing the pressure measurement data and/or the at least one breathing performance indicator, for example by comparing to previous values and/or to suitable threshold values, and providing the user with one or more automated suggestions on how to improve their usage of the device and optimise their training.

In accordance with a fourth aspect of the invention, there is provided a computer system for monitoring the breathing performance of a user, the computer system including at least one processor for executing program instructions configured to:
receive input from a user relating to a target resistance level;
receive pressure measurement data from the intelligent adaptor according to the first or second aspects of the invention;
determine at least one breathing performance indicator of the user based at least on a comparison of the target resistance level with the pressure measurement data;
display the pressure measurement data and/or the at least one breathing performance indicator on one or more display screens of a display device for viewing by the user.

The at least one processor can additionally be configured to analyse the pressure measurement data and/or the at least one breathing performance indicator, for example by comparing to previous values and/or to suitable threshold values, and can provide the user with one or more automated suggestions on how to improve their usage of the device and optimise their training.

In accordance with a fifth aspect of the invention, there is provided a system for determining a user's isometric respiratory muscle strength, the system comprising:
an intelligent adaptor having a first end and a second end connected by a passageway, and a selectively removable plug at least partially located within the first end to substantially occlude the passageway; wherein the second end is configured to be received into a mouth of a user, such that in use, a substantially sealed chamber is created between at least the user's lungs and the plug, thereby enabling the user to generate a pressure within the passageway upon activation of their respiratory muscles under isometric conditions;
the intelligent adaptor further comprising a control system, the control system comprising:
   a pressure sensor configured to measure the pressure generated within the passageway at pre-defined increments of time during activation of the user's respiratory muscles under isometric conditions; and,
   a transceiver configured to receive pressure measurement data from the pressure sensor, and transmit the data to a computing device for analysis.

In use, the volume of air between the user's lungs and the plug may remain substantially constant since there is no, or negligible, air flow between the user's lungs and the plug. The user can therefore generate, in use, a pressure increase or decrease within the passageway upon activating their respiratory muscles under isometric conditions (i.e., by expanding or contracting their lungs for a constant volume of air).

In some embodiments, the plug may comprise a pin-hole extending axially through the plug. The pin-hole may be a pressure vent, such that in use, a small amount of user-generated pressure can leak through the plug. In use, the presence of the pin-hole can help to prevent the user from generating any pressure using their cheek muscles (e.g., the buccinator muscle) and/or prevent glottic closure (i.e., closure of the vocal folds/cords), thereby helping to ensure any pressure within the passageway is generated, as much as possible, by the user's respiratory muscles.

In use, the pressure measurements obtained when the plug is present can be used to determine the user's maximum respiratory pressure, which can be indicative of the maximum strength of the user's respiratory muscles. Herein, “maximum respiratory pressure” is understood to mean the maximum expiratory pressure and/or the maximum inspiratory pressure generated under isometric conditions, and “respiratory muscles” is understood to mean the expiratory and/or the inspiratory muscles, respectively.

In embodiments, the computing device can comprise a software application, the software application may be configured to:
receive pressure measurement data from the pressure sensor;
identify a maximum respiratory pressure based on the received pressure measurement data; and
determine at least one resistance level for the user based, at least in part, on a comparison of the maximum respiratory pressure identified, or a percentage thereof, with resistance loads associated with a resistance valve located within a chosen breathing training device.

The chosen breathing training device can either be an inspiratory training device or an expiratory training device. The resistance load may be adjusted in use to increase or decrease the load provided by the resistance valve, thereby increasing or decreasing the air pressure required to move the resistance valve from a first to a second configuration.

The at least one resistance level may be determined based on a percentage of the maximum respiratory pressure generated, for example, 30%, 40%, and/or 50%. However, it will be appreciated that the at least one resistance level may be based on any other suitable percentage(s) of the maximum respiratory pressure generated.

In embodiments, the at least one resistance level can additionally be determined based on user input regarding at least their previous and current lung health, including activity levels and smoking habits, any previous breathing training sessions, and future goals.

In embodiments, the software application can be further configured to display the at least one resistance level, optionally along with at least one recommended training programme (or plan or guide), e.g., the number breaths per training session and/or the number of training sessions per day. In embodiments, a target resistance level may be selected from the at least one resistance level based on user input, such that any future training sessions can be initiated based on the target resistance level. In this manner, the computing device can act as a coach to provide a user with guidance on selecting an appropriate resistance level upon which to commence training, thereby helping to optimise training to ensure continued progression.

In accordance with a sixth aspect of the invention, there is provided a method of determining a user's isometric respiratory muscle strength using the system according to the fifth aspect of the invention, the method comprising:
placing the second end of the intelligent adaptor into a user's mouth, such that a substantially sealed chamber is created between at least the user's lungs and the plug, thereby enabling the user to generate a pressure within the passageway upon activation of their respiratory muscles under isometric conditions;
generating a pressure within the passageway between the user's lungs and the plug;
measuring the air pressure within the passageway at pre-defined increments of time;
transmitting the pressure measurements to a computing device;
determining a maximum respiratory pressure generated by the user based on received pressure measurement data; and,
determining at least one resistance level for the user based, at least in part, on a comparison of the maximum respiratory pressure generated, or a percentage thereof, with resistance loads associated with a resistance valve located within a chosen breathing training device.

The method can further comprise displaying the pressure measurements and/or the maximum respiratory pressure generated and/or the at least one resistance level on one or more display screens of a computing device for viewing by the user. In embodiments, the method may further comprise displaying one or more training programmes (or plans or guides) associated with the or each resistance level (e.g., the number breaths per training session and/or the number of training sessions per day).

In embodiments, the method may further comprise selecting a target resistance level from the at least one resistance level based on user input. In embodiments, the selected target resistance level can be manually or automatically input into the computing device prior to commencing a training session in accordance with the first, second, third and/or fourth aspects of the invention.

Whilst the invention has been described above, it extends to any inventive combination set out above, or in the following description or drawings.

### BRIEF DESCRIPTION OF THE DRAWINGS

Specific embodiments of the invention will now be described in detail by way of example only and with reference to the following drawings, in which:

FIG. 1a shows various perspective views of an intelligent adaptor according to aspects of the present invention;

FIG. 1b shows a cross-sectional view of the FIG. 1a intelligent adaptor;

FIG. 1c shows an exploded view of the FIG. 1a intelligent adaptor;

FIG. 1d shows a perspective cross-sectional view of the FIG. 1a intelligent adaptor;

FIG. 2 shows a perspective view of the intelligent adaptor attached to a breathing training device according to aspects of the present invention;

FIG. 3 shows an example graph of air pressure measurements over time to demonstrate the detection of inhalation and exhalation start and end points;

FIG. 4 shows a further example graph of air pressure measurements over time to demonstrate the detection of different resistance levels;

FIG. 5 shows a graph of air pressure measurements demonstrating successful and unsuccessful inhalations;

FIG. 6 shows a flow diagram of a computer algorithm according to aspects of the present invention;

FIG. 7a shows an example display on a mobile phone screen of a number of breathing performance indicators from a breathing training session;

FIG. 7b shows a further example display on a mobile phone screen of one breathing performance indicator monitored over time;

FIG. 8A shows various perspective views of a plug for use with the FIG. 1a-d intelligent adaptor;

FIG. 8B shows front and rear perspective views of an alternative plug for use with FIG. 1a-d intelligent adapter;

FIG. 9 shows a system comprising the FIG. 8 plug attached to the FIG. 1a-d intelligent adaptor according to further aspects of the present invention;

FIG. 10a shows an example display on a mobile phone screen of results from an isometric respiratory muscle strength test using the FIG. 9 system;

FIG. 10b shows an example display on a mobile phone screen of suggested resistance levels based, at least in part, on the FIG. 10a test results; and, FIGS. 11a and 11b show example pop-up displays on a mobile phone screen providing further information regarding the suggested resistance levels.

### DETAILED DESCRIPTION OF EMBODIMENTS

The present invention relates to an intelligent adaptor which can be attached to the mouthpiece of a breathing training device for monitoring a number of breathing performance indicators of a user during a training session. This is achieved by providing the intelligent adaptor with a pressure sensor to measure the pressure generated by the user at pre-defined increments of time, and then transmitting these measurements to a computing device where they are analysed to determine various breathing performance indicators. The user can monitor their performance during a single training session, and also monitor their performance across a number of training sessions. The ability to monitor their progress can allow the user to make any required adjustments to their training plan so they are able to reach their target performance.

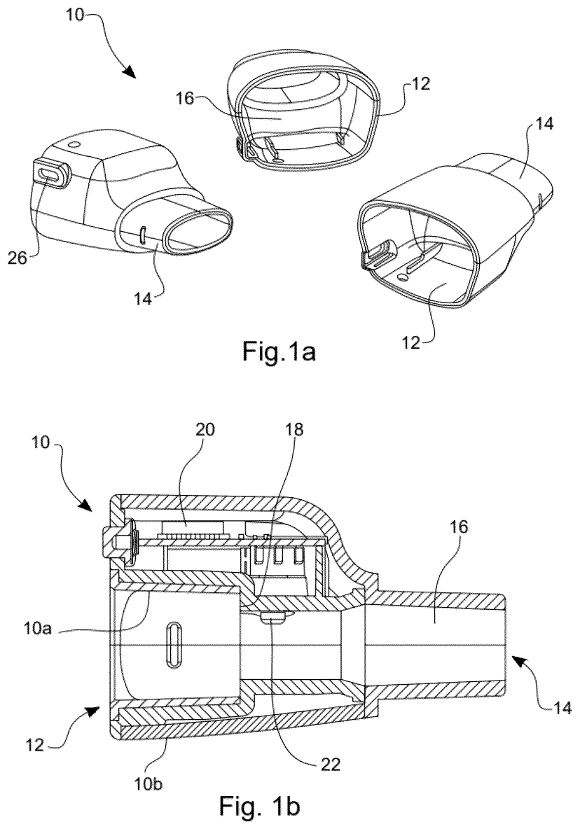

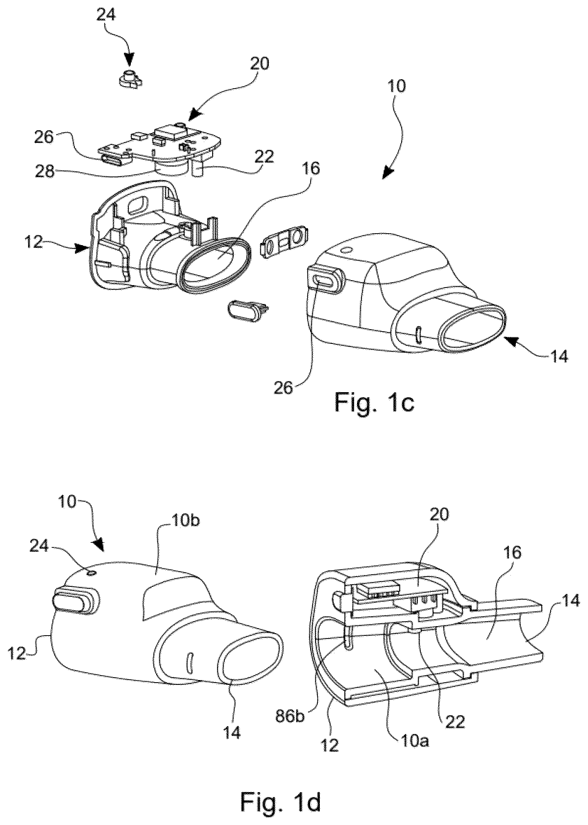

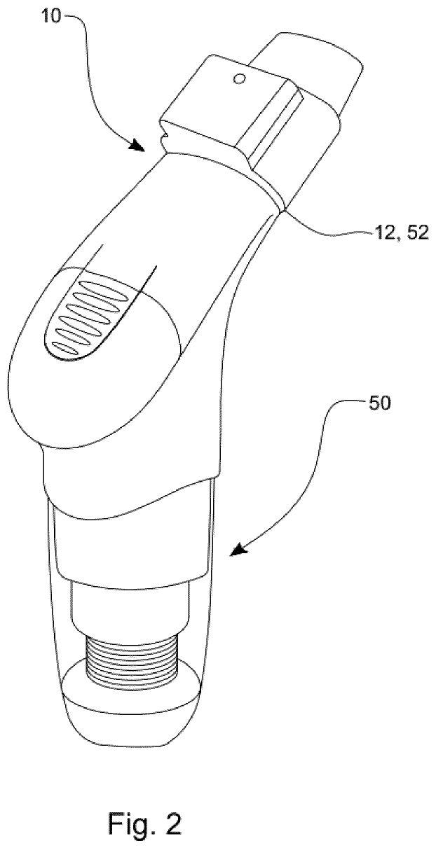

FIGS. 1a-1d show an adaptor 10 for attaching to a breathing training device, and FIG. 2 shows a system whereby the adaptor 10 is attached to a breathing training device 50. The adaptor 10 comprises a first end 12 which attaches to the mouthpiece 52 of the breathing training device 50, and a second end 14 which a user breathes into, in use. The first and second ends 12, 14 are connected by a passageway 16. The device 50 comprises a resistance valve (not shown) which moves between a closed and an open position when a threshold air pressure value is crossed. The resistance valve can be adjusted such that the threshold air pressure is increased or decreased between pre-set limits to make it harder or easier to move the valve. It is to be appreciated that adaptor 10 may be formed as single or multiple moulded parts, for example having the main body with mouthpiece and separate airway, as shown in FIG. 1c or, for example, having three parts comprising the main body, mouthpiece and airway (not shown).

The device 50 in the present example is an inspiratory training device, meaning that the resistance valve provides a resistance load when the user inhales. There is no or minimal resistance load provided during the exhale. When the user inhales, the pressure within the device decreases. The air pressure must drop below a threshold value to move the valve from the closed to the open position, and allow air to freely flow between the device and the user's lungs.

Adjusting the resistance valve adjusts the resistance load provided by the valve, and therefore adjusts the threshold pressure value. Increasing the threshold pressure makes it harder to open the valve (more effort required as a larger pressure drop is required), whereas decreasing the threshold pressure makes it easier to open the valve (less effort required as a smaller pressure drop is required).

The resistance load can be varied incrementally, such that each increment can span a range of resistance loads. Each range therefore can define a resistance level, with the threshold value being the minimum air pressure drop required to enter a level and open the valve. As will be described further below, prior to a training session commencing, the user can set a target resistance level depending on their abilities and training goals.

The adaptor 10 further comprises a control system 20. The control system 20 is powered by a rechargeable power source, such as a battery 28. The adaptor 10 also comprises a power input 26, which is configured to connect to an external power supply. For example, the power input 26 can be a micro-USB port configured to connect to a 5 volt USB power adaptor to charge the battery. The adaptor may also include a visual indicator, for example, to illustrate that it is operational or switched on. This may be, for example, an LED that shines through a hole in the main body of the adaptor or an LED that shines through the material of the main body.

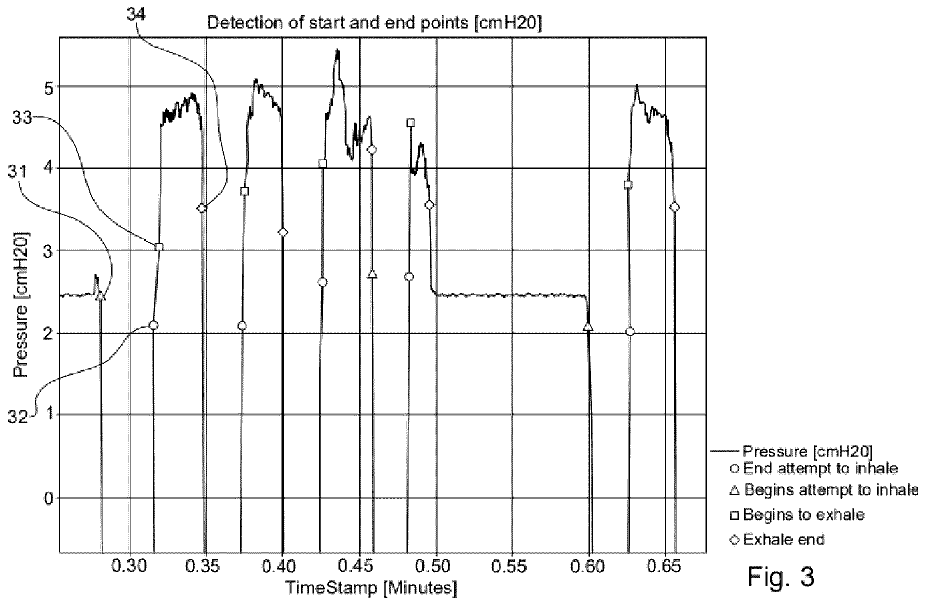

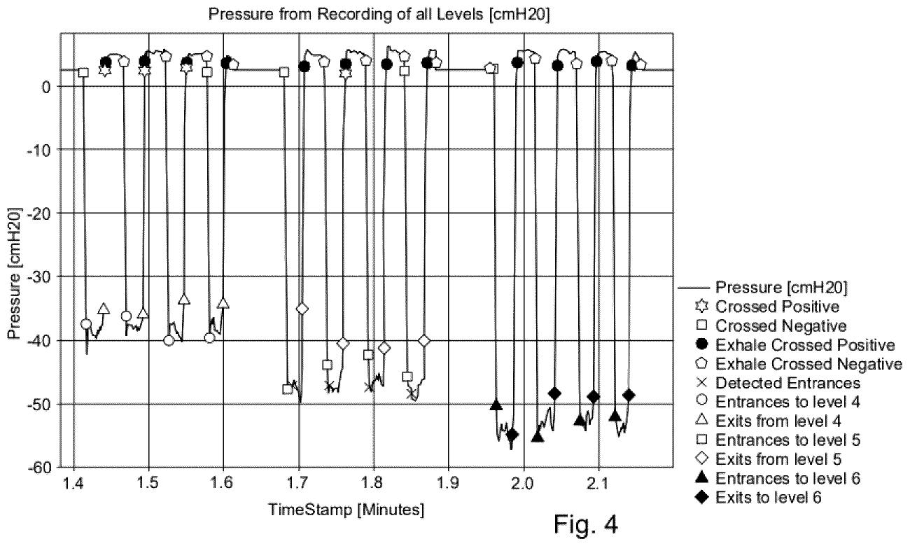

In use, a sealed chamber is formed between the user's lungs, the passageway 16, the mouthpiece 52, and all the way to the resistance valve. The control system 20 comprises a pressure sensor 22 which measures the user-generated pressure within the passageway 16 at pre-defined increments of time during a training session. Specifically, the pressure sensor 22 measures the user-generated pressure within the sealed chamber. The pressure sensor 22 measures the air pressure generated by the user at the location of the sensor 22 in the passageway 16. In this example, the pre-defined increments of time are 42 ms, which provides a sampling rate of around 24 Hz. As shown in FIGS. 3 and 4, and as will be described further below, the pre-defined increments of time are sufficient to allow the pressure sensor 22 to adequately detect when a user attempts to open the valve (in cases where the time to open the valve is greater than a minimum threshold time), and also to adequately detect when a user moves between resistance levels. The error margin for time-related variables is equal to the size of the increments (in this case 42 ms). In embodiments, the error margins for the pressure sensor measurements are not significant, and can be disregarded.

The control system 20 further comprises a transceiver 24 which in use receives the pressure measurement data from the pressure sensor 22, and transmits this data to a computing device for analysis. The computing device is preferably a smartphone with Bluetooth functionality, and the transceiver 24 is a wireless Bluetooth device. The analysis performed by the smartphone involves determining several breathing performance indicators of the user, as will be discussed below.

The smartphone comprises a software application, referred to as an app. As will be described in more detail below, prior to the training session commencing, the user can inform the app of their target resistance level. During the session, the app receives the pressure measurement data from the pressure sensor 22, and uses this data, to determine various breathing performance indicators of the user, including how many times the user crossed the threshold pressure into their target resistance level.

A non-limiting implementation of the adaptor 10 will now be described in use.

To start, the user opens the app on their smartphone (or first downloads the app from the appropriate app marketplace). They will be given the option to sign-in if they already have an account, or if not, they will be given the option to create an account.

The account set-up procedure will require the user to input a number of details, such as personal information (e.g., name, email address, date of birth, gender, height, and weight) and respiratory health information (e.g., activity levels, smoking habits, and any respiratory conditions, such as asthma or chronic obstructive pulmonary disease (COPD)). The user will also be asked to input information about their breathing training device (e.g., the model, resistance type, and colour) and reasons for using the device (e.g., fitness or rehabilitation, the intended number of sessions per day, and the intended number of training days per week), and will then be taken through the process to pair their intelligent adaptor 10 with the app via the Bluetooth functionality.

The app will then take the user through the process for taking their first recording, including how to adjust the resistance load of the device 50 and inputting their target resistance level.

Once the user has signed-in to their account, or created an account and gone through the set-up process, the user can begin to record their training sessions.

Prior to commencing a training session, the user adjusts the resistance valve on the device 50 to their target resistance level, and enters their target resistance level into the app. The user then places the second end 14 of the adaptor 10 into their mouth, forms a seal around the second end 14 using their lips, and begins breathing through the device. The user also indicates to the app that they want to start recording the training session, which then enables the app to begin plotting the pressure measurements sent from the pressure sensor 22 as the user breathes in and out. During their inhalations, the user attempts to provide a sufficient air pressure drop within the device to cross the threshold and open the valve at their target resistance level.

As the user generates pressure within the passageway 16 during attempted inhales and exhales, the pressure sensor 22 takes a pressure reading every 42 ms. The pressure measurements are sent to the app via the transceiver 24, and plotted on the smartphone screen in real-time. FIGS. 3 and 4 show example graphs of the pressure readings (y-axis) against time (x-axis), zoomed in to demonstrate the ability of the app to detect the start and end points of each inhale and each exhale, the entry and exit of different resistance levels.

FIG. 3 shows that a sampling rate of 24 Hz (42 ms) is sufficient to detect the moments when the user begins inhaling 31, ends inhaling 32, begins exhaling 33, ends exhaling 34, and then begins inhaling again 31. FIG. 4 then shows that the sampling rate of 24 Hz is sufficient to additionally detect when a user enters and exits different resistance levels. Between around 1.4-1.6 minutes, the algorithm detects that the air pressure generated by the user during an inhalation has crossed the threshold air pressure value to enter resistance level 4, and then exit resistance level 4 again as the air pressure generated begins to increase. In this example, resistance level 4 is defined by a drop in air pressure of around 35-40 cmH₂O. Furthermore, between around 1.7-1.9 minutes, the algorithm detects that the air pressure generated by the user during an inhalation has crossed the threshold air pressure value to enter resistance level 5, and then exit resistance level 5 again as the air pressure generated begins to increase. In this example, resistance level 5 is defined by a drop in air pressure of around 40-45 cmH₂O. Finally, between around 1.95-2.15 minutes, the algorithm detects that the air pressure generated by the user during an inhalation has crossed the threshold air pressure value to enter resistance level 6, and then exit resistance level 6 again as the air pressure generated begins to increase. In this example, resistance level 6 is defined by a drop in air pressure of around 50-55 cmH₂O. As will be described in more detail below, the algorithm compares the above readings with the user's target resistance level, and alerts the user each time they enter their target resistance level and open the valve. A tally is displayed on the screen alongside the pressure plot, which shows the number of successful inhalations and the total number of inhalation attempts.

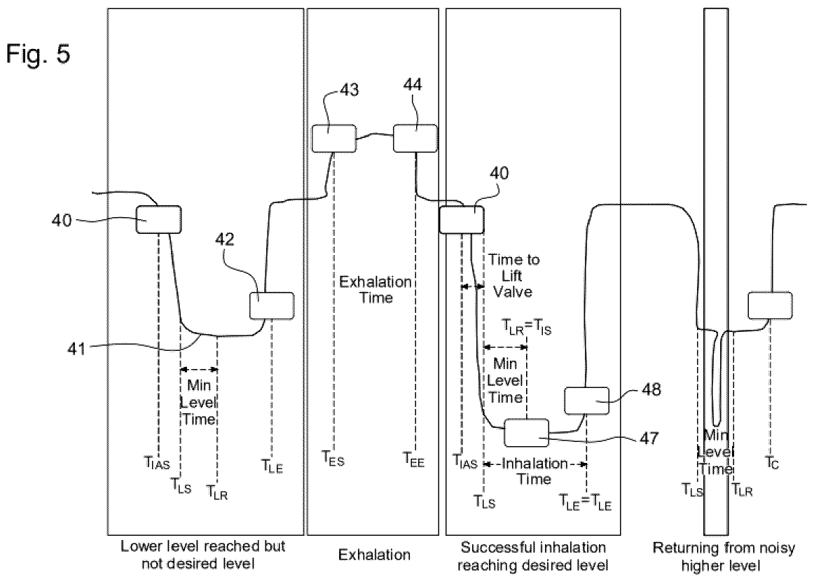

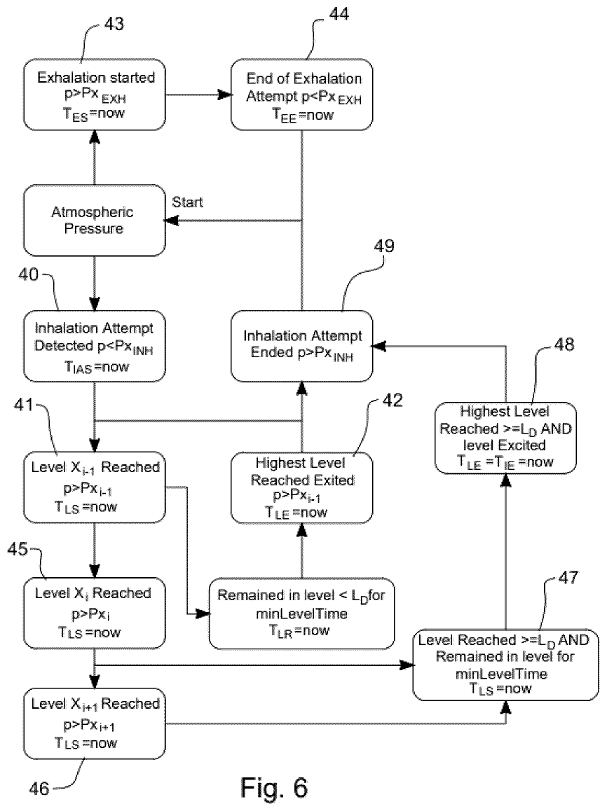

FIG. 5 shows a further example graph of part of a user's training session, and FIG. 6 shows the process to determine if an inhalation attempt should be recorded as successful. The adaptor 10 and device 50 begin at atmospheric pressure. At step 40, the user begins an attempt to inhale. When the pressure drops by at least a predefined amount below zero (the predefined amount being dependent on the resistance level), the algorithm registers that an inhalation attempt has begun, and adds 1 to the total number of inhalation attempts. During this attempt, the user progresses through different resistance levels until a maximum air pressure drop is reached at step 41. In this example, the drop in air pressure is not sufficient to reach the target level (L₀), however the user is able to maintain this drop in air pressure for at least a minimum amount of time, which allows the algorithm to detect that a resistance level lower than the target level was reached. At step 42, when the air pressure starts to increase, the inhalation attempt is recorded as being unsuccessful because the target level was not reached and so the valve was not opened.

At steps 43 and 44, the user is beginning and ending an exhalation. The algorithm detects that an exhalation has begun when the pressure increases to at least a predefined (level-dependent) amount above zero, and ended when the pressure drops to below the predefined (level-dependent) amount above zero. This data is not used for determining the breathing performance indicators. However, this data is still collected and monitored, as in some scenarios a user may benefit from a notification advising them to take longer exhales to avoid hyperventilating.

The user then returns to step 40 and begins another attempt to inhale. The algorithm detects that another inhalation attempt has begun when the pressure drops by at least a predefined (level-dependent) amount below zero, and adds 1 to the total number of inhalation attempts. During this attempt, the air pressure drop caused by the inhalation attempt either falls within the target resistance level (step 45), or it falls within a greater resistance level (step 46). Either way, the pressure drop is sufficient to open the resistance valve. At step 47, if the amount of time the user remains within the target or greater resistance level spans a minimum amount of time, then the inhalation attempt is recorded as being successful at step 48. The algorithm then adds 1 to the number of successful inhalations.

If the user is able to provide a sufficient air pressure drop during an inhalation to reach the target level, but is unable to maintain the required air pressure for the minimum amount of time, and so the valve was not opened (or not fully opened for long enough), then the inhalation attempt is recorded as being unsuccessful.

The end of the inhalation attempt is detected at step 49, as being when the air pressure increases to less than a predefined (level-dependent) amount below zero.

As such, to record an attempt as successful, the user must both cross the threshold into their target resistance level and remain within that level for a minimum amount of time. If either, or both, of these conditions are not met, the attempt is recorded as unsuccessful.

In the above example, the minimum amount of time is set to be 136.36 ms. Therefore, if a user maintains a pressure within a particular resistance level for at least 136.36 ms, the algorithm registers that that resistance level was the highest level reached. The algorithm can then determine how the highest level reached compares to the target resistance level.

During the training session, the data recorded includes:
the number of successful inhalations;
the number of inhalation attempts;
the amount of time taken to reach the threshold pressure required to open the valve during the previous successful attempt;
the inhalation time of the previous successful inhalation, and the average inhalation time;
the maximum pressure reached during the previous inhalation, and the maximum inhalation pressure achieved overall;
the exhalation time of the previous exhalation, and the average exhalation time;
the maximum exhalation pressure reached;
the average breathing rate per minute;
the average time to open the valve;
the last resistance level reached, and the highest resistance level reached;
the total recording time;
the average pressure time product (PTP) achieved and/or the total pressure time product (PTP) achieved during the training session; and,
the type of activity (which the user inputs at the beginning of a session).

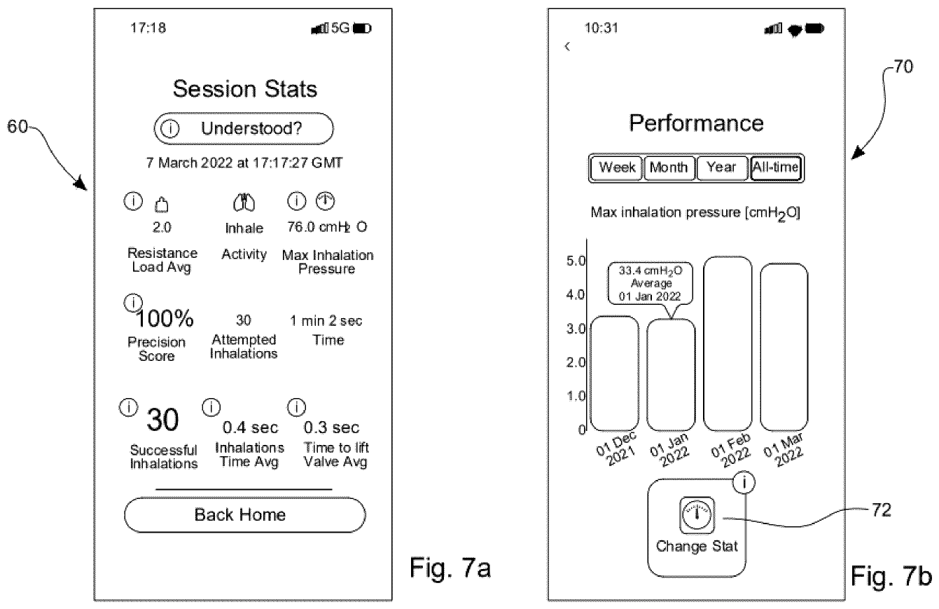

At least some of the above-recorded data is displayed on the app in real-time for viewing by the user. For example, FIG. 7a shows an example display screen 60 on a smartphone device, which shows the following information:
the resistance load average (the average resistance level reached per breath in a session; the higher the value, the more effort that was required per breath);
max inhalation pressure (the maximum air pressure reached (i.e., the maximum drop in pressure) in a training session; the higher the value, the more effort that was required);
precision score (the ratio of how many times the target resistance level was successfully reached compared to the number of unsuccessful attempts);
successful inhalations (how many times the target resistance level was successfully reached);
inhalation time average (how long the user sustained an inhalation whilst within the target resistance level; the higher the value, the longer the controlled inhalation against a resistance load); and,
Time to lift valve average (how quickly the user could generate enough of a pressure drop to open the valve and breathe in through the device).

As such, inputting the target resistance level into the app prior to commencing a training session, will allow the algorithm to notify the user in real-time when they have reached their target resistance level, and also count the number of times they have reached that level. The algorithm also analyses the pressure measurement data and/or the at least one breathing performance indicator, for example by comparing to previous values and/or to suitable threshold values, and can provide the user with one or more automated suggestions on how to improve their usage of the device and optimise their training. The software application can provide one or more automated suggestions based on, but not limited to, the following scenarios (or combinations thereof):
Discouraging hyperventilation by measuring the length of their non-loaded (without resistance) exhalations or inhalations after each loaded (with resistance) breath, and if this is consistently below or above a specific threshold, sending a reminder to the user to take longer or shorter non-loaded exhalations or inhalations.
Ending a session at the appropriate time by using a running count of the number of successful inhalations or exhalations, and comparing this to a user-defined training protocol. When the number of successful attempts matches the number defined in the training protocol, the user is reminded to end their session, so they do not do too many or too few breaths.
Ensuring an appropriate breath length by using the length of the loaded inhalations or exhalations and comparing this with an optimal length, and if this is consistently too short or too long, notifying the user to try to increase or decrease the length of their loaded inhalations or exhalations, respectively.
Ensuring an appropriate training level by using the average length of the loaded inhalations or exhalations and comparing this with a threshold length, and if this is found to be longer or shorter than the threshold length, the user can be notified to move up or down a resistance level, respectively.
Discouraging ineffective training by using the time taken to reach a threshold pressure, as well as the time taken for the pressure to return to atmospheric pressure, to identify how tired the user may be, and if the user is considered to be tired or struggling, suggesting that the user ends the session early.
Encouraging proper breathing technique by using the time taken to reach the threshold pressure required to move the valve and comparing this to a threshold amount of time, and if the time taken is found to be too slow, prompting the user to increase the sharpness of their breath to produce sharper, more powerful, increases in pressure.
Ensuring an appropriate training level by using the average time taken to reach the threshold pressure required to move the valve, and comparing this with a threshold amount of time, and if this is found to be longer or shorter than the threshold amount, the user can be notified to go down or up a resistance level, respectively.
Ensuring an appropriate training level by using the average pressure across all inhalation attempts during a training session, and comparing this to a threshold value, and if it is found to be greater than or less than the threshold value, the user can be notified to go up or down a resistance level, respectively.

In this manner, the app can coach the user on the correct usage of their chosen respiratory muscle trainer to help optimise their training.

FIG. 7b shows a further example display screen 70 on a smartphone device, which allows the user (or another person, such as a medical professional) to monitor their progress over a period of time, such as a week, a month, a year, or over all-time. The statistics displayed can be changed by pressing the button 72 at the bottom of the screen. In the example shown, the user is viewing their max inhalation pressure statistics over all-time, averaged per month. If the user selects to view their progress per month, the chosen statistic is displayed as an average per day. FIG. 7b shows that the user's max inhalation pressure (i.e., the maximum pressure drop recorded) has dramatically improved between January and February of that year.

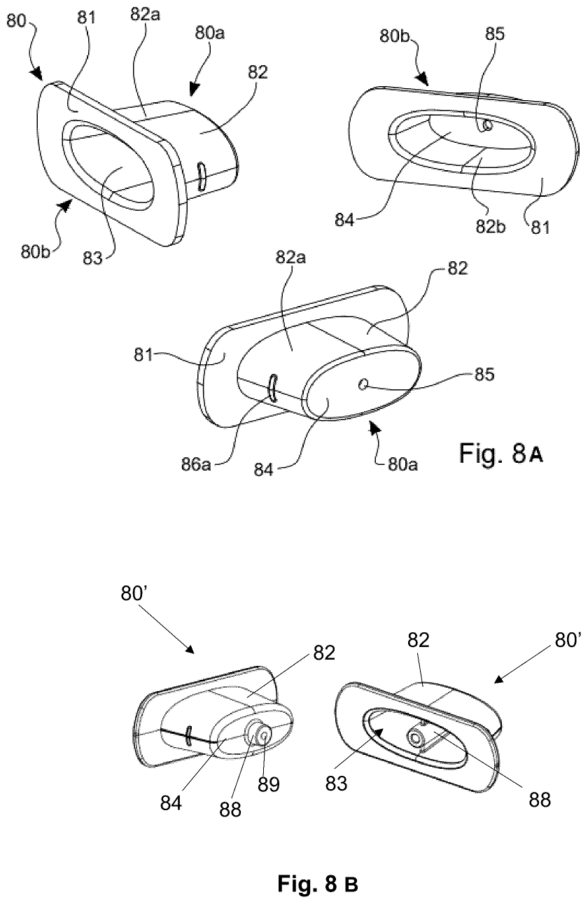

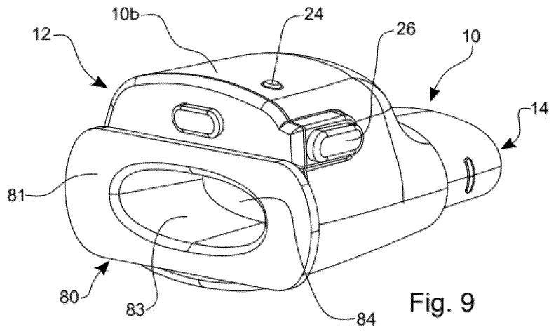

FIG. 8A shows a selectively removable plug 80 for use with the adaptor 10, and FIG. 9 shows a system whereby the plug 80 is attached to the adaptor 10 according to some aspects of the invention. The adaptor 10 can be attached, in use, to either the device 50 or the plug 80. However, it will be appreciated that in some examples, the adaptor may be configured to attach, in use, to both the device 50 and the plug 80, such that the plug 80 can form an interface between the adaptor 10 and the device 50.

In some examples, the plug 80 can form a “top hat” shape, as shown in FIG. 8. In such examples, the plug 80 comprises a hollow axially extending portion 82 having a first end 80a and a second end 80b and defining a passageway 83. The first end 80a comprises a radial base section 84 having a pin-hole 85 extending axially through the base section 84. The cross-sectional area of the pin-hole 85 is significantly smaller than the cross-sectional area of the passageway 83 (and also passageway 16).

The second end 80b comprises a rim or flange 81 which extends radially outwardly from the axially extending portion 82. In use, the flange 81 acts as a stop to help limit the distance the axially extending portion 82 can be inserted into the passageway 16 during assembly. Furthermore, the plug 80 is configured such that a user can grasp the flange 81, in use, to separate the plug 80 from the adaptor 10 (e.g., if it is desired to instead attach a device 50 to begin a training session).

The axially extending portion 82 comprises an outer surface 82a and an inner surface 82b, whereby the inner surface 82b defines the passageway 83. Furthermore, the adaptor 10 comprises an inner surface 10a and an outer surface 10b, whereby the inner surface 10b defines the passageway 16 (shown in FIGS. 1b and 1d). The outer surface 82a is complementary in size and shape to the inner surface 10a.

In particular, the cross-sectional dimensions of the outer surface 82a are slightly greater than the corresponding cross-sectional dimensions of the inner surface 10a, such that during assembly, the axially extending portion 82 can be push-fit into the passageway 16 at the first end 12 until the flange 81 contacts the first end 12 of the adaptor 10, thereby preventing further movement into the passageway 16.

Furthermore, as shown in FIG. 1b, the passageway 16 comprises a shoulder 18, against which the first end 80a of the plug 80 can abut after assembly. The presence of a shoulder 18 can help to prevent the plug 80 (or alternatively the mouthpiece 52) from contacting and potentially damaging the pressure sensor 22 during assembly.

The outer surface 82a and the inner surface 10a also comprise interlocking features which can engage when the axially extending portion 82 is fully inserted into the passageway 16 (i.e., when the flange 81 contacts the first end 12 of the adaptor 10, and/or the base section 84 contacts the shoulder 18). For example, outer surface 82a comprises a projection 86a which is configured to engage with a complimentary recess 86b on the inner surface 10a (shown in FIG. 1d). As such, the plug 80 can be retained within the passageway 16 by engagement between the projection 86a and the complimentary recess 86b.

Once assembled, passageway 16 and passageway 83 are substantially co-axial, and the plug 80 substantially occludes the first end 12 of the passageway 16, thereby substantially restricting or blocking any flow of air through the passageway. The plug 80 can be used in combination with the adaptor 10 when performing an isometric respiratory muscle strength test, such as a maximum expiratory pressure (MEP) test or a maximum inspiratory pressure (MIP) test, which aims to determine the maximum strength of the user's respiratory muscles (i.e., their inspiratory muscles and/or expiratory muscles).

Prior to commencing a training session, the plug 80 can be removed from the passageway 16, for example, by disengaging the projection 86a from the complimentary recess 86b and sliding the axially extending portion 82 out of the passageway 16 (e.g., by grasping the flange 81 and applying a pulling force). The adaptor 10 can then be attached to the device 50 and used in the manner described above. An alternative embodiment of a plug 80′ is shown in FIG. 8B in which the same features are given the same reference numerals and only the differences will be discussed herein. In this embodiment, a cylindrical inner part 88 is provided through passageway 83, being moulded to the inner surface of the extending portion 82 and terminating in pin hole 89 beyond base section 84.

A non-limiting implementation of the adaptor 10 will now be described in use when combined with the plug 80.

The user can start by opening the app and signing-in to their account (or setting one up) and going through the process to pair their breathing training device with the app via the Bluetooth functionality, in the same manner as described above. The user can then select the option to perform a maximum respiratory pressure test, which effectively provides a measure of the maximum strength of the user's respiratory muscles. In examples, if the paired device is an inspiratory training device, such as device 50, the user may be given the option to perform a MIP test. As such, the test will provide an effective measure of the maximum strength of the user's inspiratory muscles. In other examples, if the paired device is an expiratory training device, the user may be given the option to perform a MEP test, thereby effectively measuring the maximum strength of the user's expiratory muscles.

Prior to commencing the MIP (or MEP) test, the user assembles the adaptor 10 and plug 80 as described above. Once assembled, the plug 80 substantially occludes (or restricts or blocks or closes) the passageway 16 at the first end 12. The user can also indicate in the app that they want to start recording the test, which enables the app to begin recording the pressure measurements received from the pressure sensor 22.

The user then places the second end 14 of the adaptor 10 into their mouth and forms a seal around the second end 14 using their lips. During each inhalation attempt, the pin-hole 85 permits a small amount of air pressure to leak through the plug 80. This can help to prevent the user from using their cheek muscles and/or help to prevent glottic closure, which can skew the results of the test and provide an inaccurate determination of the user's maximum inspiratory (or, in some examples, expiratory) muscle strength.

During each inhalation attempt, the air pressure in the passageway 16 can be measured by the pressure sensor 22 (i.e., the pressure generated in the absence of air flow through the passageway) at pre-defined increments of time during the test, and the data is transmitted to the app via the transceiver 24. In some examples, the pressure data can be plotted on the smartphone screen in real-time.

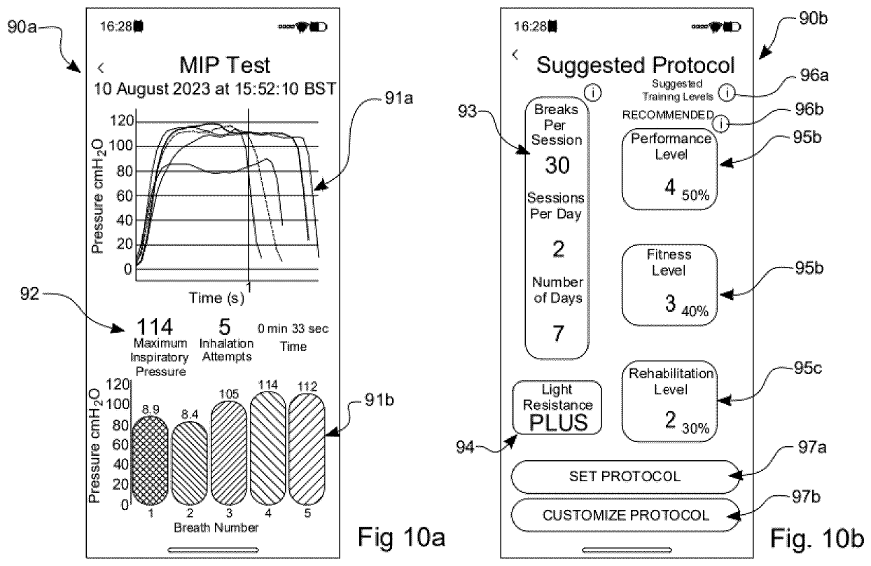

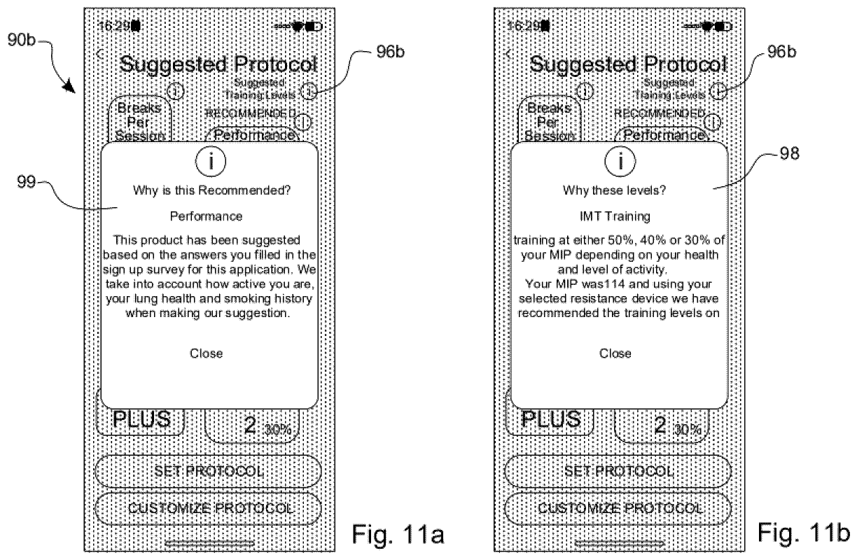

FIG. 10a shows an example display screen 90a on a smartphone device to allow the user to view the results of their MIP (or MEP) test. In this example, the user has made five inhalation attempts, each represented by a different line on graph 91a.

Graph 91a plots the pressure readings (y-axis) measured in cmH₂O against time (x-axis) measured in seconds. The data is analysed by the app, and the maximum inspiratory pressure is identified for each inhalation. Graph 91b then plots the MIP identified (y-axis) measured in cmH₂O against inhalation or breath number (x-axis). Graph 91b effectively represents the maximum strength of the user's inspiratory muscles during each inhalation attempt under isometric conditions (i.e., changes in pressure for a constant volume). The largest MIP recorded across all inhalation attempts can also be numerically displayed, as represented by reference 92. In this example, the largest MIP achieved by the user during the test is 114 cmH₂O.

Once the MIP test is complete, the results are analysed in conjunction with information about the breathing training device (e.g., the model, resistance type, and resistance levels), information provided by the user during account set-up (e.g., the user's respiratory health, activity information, smoking history), and any other relevant information (e.g., previous breathing training sessions, either based on user input or based on data collected during the previous training sessions, including number of sessions and the number of successful attempts at different target resistance levels).

The app can then determine at least one resistance level and at least one associated training programme for the user based on a comparison of the maximum inspiratory pressure identified 92, or a percentage thereof, with the resistance loads provided by the resistance valve located within the breathing training device, such as device 50.

FIG. 10b shows an example display screen 90b on a smartphone device to allow the user to view a number of training protocols 95a, 95b, 95c. Each training protocol 95a, 95b, 95c comprises a resistance level and an associated training programme 93 using the breathing training device indicated in section 94, which in this example is the Powerbreathe Plus IMT device having a light resistance (i.e., 17 cmH₂O-98 cmH₂O).

In the present example, the app recommends three training protocols: performance 95a; fitness 95b; and, rehabilitation 95c. Each of the training protocols 95a, 95b, 95c comprises a different target resistance level, and may also comprise a different training plan 93, depending on the user's current lung health and training goals. For example, if a user has no previous breathing training experience, the app may recommend the rehabilitation protocol (particularly if the user has weakened or damaged lungs due to illness or injury). In other examples, if the user has some previous breathing training experience, the app may recommend the fitness or performance protocols (depending on the amount of previous training, the intensity of the training, and overall lung health).

The training programme 93 provides details of the recommended number of breaths per training session, the recommended number of sessions per day, and the recommended number of training days per week. It will be appreciated that alternative or additional details may be included in the training programme 93. The training programme 93 may be automatically modified depending on the user's current lung health and training goals. For example, if a user has a significant amount of breathing training experience, the app may modify the training programme 93 to enable the user to maintain their current lung strength rather than progressively building strength (e.g., by reducing the number of training days, such that the user is training every other day instead of every day).

In some examples, the user may be periodically prompted to retake the MIP (or MEP) test, so that their training protocol (i.e., the resistance level and training programme) can be modified, if needed, to ensure training progression or maintenance at their current level.

In this example, the app recommends that performance training protocol 95a would be most suitable for the user based on their current lung health and future goals. The performance training protocol 95a suggests that the user commence their training sessions at a target resistance level of 4 (approximately 49 cmH₂O-57 cmH₂O for the paired device 94), which is equivalent to training at around 50% of their MIP. The app further recommends a training programme 93 involving training twice per day, every day, and aiming for 30 breaths per training session. In other examples, the app may alternatively recommend training protocols 95b or 95c, in which case these options would be highlighted to the user.

If the user wishes to follow one of the recommended training protocols 95a, 95b, 95c, they can select the desired protocol and then select “Set Protocol” 97a. In some examples, the user may wish to proceed with a different training protocol, such as a different target resistance level and/or a different training programme. In these cases, the user can select “Customise Protocol” 97b, where they will be given the option to modify the target resistance level and/or modify the training programme (e.g., manually change the number of sessions per day).

FIGS. 11a and 11b show example displays 98, 99 which may appear on the display screen 90b of a smartphone device upon selecting the “information” symbols 96a and 96b. If the user selects the “information” symbol 96a, pop-up 98 will appear on the screen to inform the user why these resistance levels were recommended. If the user selects the “information” symbol 96b, pop-up 99 will appear on the screen to inform the user why the associated training programmes have been suggested.

In use, after completing the MIP test and prior to commencing a training session (which may be based on a selected training protocol), the user detaches the plug 80 from the adaptor 10, and then attaches the adaptor 10 to the mouthpiece 52 of a breathing training device, such as device 50. The user can then adjust the resistance valve on the device to the selected target resistance level and also enter their target resistance level into the app. In some examples, the target resistance level may be automatically entered based on the selected training protocol. The user can then commence a training session in the same manner as described above.

Whilst the principle of the invention has been illustrated using exemplary embodiments, it will be understood that the invention is not limited to exemplary embodiments and that the invention may be embodied by other variants defined within the scope of the appended claims.

1. An intelligent adaptor for a pressure threshold breathing training device, the adaptor comprising:
a first end configured to attach to a mouthpiece of a the pressure threshold breathing training device;
a second end configured to be received into a mouth of a user, such that a seal is created in use around the second end of the adaptor;
the first and second ends being connected by a passageway, the passageway being configured to provide a sealed chamber, in use, between at least the lungs of the user and the mouthpiece of the pressure threshold breathing training device, such that if a measured pressure within the passageway of the adaptor crosses a threshold value, a resistance valve located within the pressure threshold breathing training device moves from a first to a second configuration to allow the user to breathe freely through the device in the second configuration;
the adaptor further comprising a control system comprising:
a pressure sensor configured to measure a user generated pressure within the passageway at pre-defined increments of time during a training session; and,
a transceiver configured to receive pressure measurement data from the pressure sensor, and transmit the data to a computing device for analysis.

2. (canceled)

3. An adaptor as claimed in claim 1, wherein the pre-defined increments of time are chosen such that the pressure sensor can adequately detect when the user begins an inhalation, ends an inhalation, begins an exhalation, and ends an exhalation using the pressure threshold breathing training device.

4-12. (canceled)

13. An adaptor as claimed in claim 1, wherein the passageway at the first end is configured to at least partially receive a selectively removeable plug, such that in use, the plug substantially occludes the passageway of the adaptor at the first end, thereby at least minimising air flow between lungs of the user and the plug, and enabling the user to generate pressure within the passageway under isometric conditions.

14. The adaptor as claimed in claim 1 in combination with:
a) the pressure threshold breathing training device; and
b) a computing device for analysis of data received from the intelligent adaptor;
wherein the intelligent adaptor is mounted onto the mouthpiece of the pressure threshold breathing training device.

15. (canceled)

16. An adaptor as claimed in claim 14, wherein a resistance load provided by the resistance valve is adjustable to increase or decrease the threshold value at which the resistance valve moves from the first to the second configuration thereby providing an adjustable threshold value.

17. An adaptor as claimed in claim 14, wherein a resistance load provided by the resistance valve is variable incrementally up to a maximum resistance load, such that each increment spans a range of resistance loads, thereby defining a series of progressively increasing resistance levels.

18. An adaptor as claimed in claim 17, wherein each resistance level comprises a threshold value which must be crossed to move the resistance valve from the first to the second configurations; and wherein the pre-defined increments of time are sufficient to allow the pressure sensor to adequately detect when a the user moves into and out of a resistance level.

19. A method of monitoring the breathing performance of a user using the intelligent adaptor, pressure threshold breathing device and computing device of claim 14, the method comprising:
inputting a target resistance level into the computing device;
placing the second end of the intelligent adaptor into the mouth of the user, such that a seal is created around the second end of the adaptor;
generating a pressure within the passageway between the user and the resistance valve;
measuring the air pressure within the passageway at pre-defined increments of time to provide air pressure measurements;
transmitting the air pressure measurements to the computing device;
determining at least one breathing performance indicator of the user based at least on a comparison of a target resistance level with the air pressure measurements; and
displaying the air pressure measurements and/or the at least one breathing performance indicator, on one or more display screens of the computing device for viewing by the user.

20. A method as claimed in claim 19, further comprising analysing pressure measurement data and/or the at least one breathing performance indicator, and providing the user with one or more automated suggestions on how to improve their usage of the pressure threshold breathing training device and optimise their training.

21. A computer system for monitoring the breathing performance of a user, the computer system including at least one processor for executing program instructions configured to:
receive input from the user relating to a target resistance level;
receive air pressure measurements from the intelligent adaptor one of claim 1;
determine at least one breathing performance indicator of the user based at least on a comparison of the target resistance level with the air pressure measurements;
display pressure measurement data and/or the at least one breathing performance indicator on one or more display screens of a display device for viewing by the user.

22. A computer system as claimed in claim 21, wherein the at least one processor is additionally configured to analyse the pressure measurement data and/or the at least one breathing performance indicator, and provide the user with one or more automated suggestions on how to improve their usage of the adaptor and optimise their training.

23. A system for determining a user's isometric respiratory muscle strength, the system comprising:
a computing device, an intelligent adaptor having a first end and a second end connected by a passageway, and a selectively removable plug at least partially located within the first end to substantially occlude the passageway of the intelligent adaptor; wherein the second end is configured to be received into a mouth of a user, such that in use, a substantially sealed chamber is created between at least lungs of the user and the plug, thereby enabling the user to generate a pressure within the passageway upon activation of their respiratory muscles under isometric conditions; and wherein,
the intelligent adaptor further comprises a control system, the control system comprising:
a pressure sensor configured to measure the pressure generated within the passageway at pre-defined increments of time during activation of respiratory muscles of the user under isometric conditions; and,
a transceiver configured to receive pressure measurement data from the pressure sensor, and transmit the data to the computing device for analysis.

24. A system as claimed in claim 23, wherein the computing device comprises a software application, the software application being configured to:
receive the pressure measurement data from the pressure sensor;
identify a maximum respiratory pressure based on the received pressure measurement data; and,
determine at least one resistance level for the user based, at least in part, on a comparison of the maximum respiratory pressure identified, or a percentage thereof, with resistance loads associated with a resistance valve located within a chosen pressure threshold breathing training device.

25. A system as claimed in claim 24, wherein the at least one resistance level and an associated training plan is additionally determined based on user input regarding at least their previous and current lung health, including activity levels and smoking habits, any previous breathing training sessions, and future goals.

26. An adaptor as claimed in claim 14, wherein the computing device comprises a software application, the software application being configured to:
receive input from the user relating to a target resistance level; thereby determining the threshold value which must be crossed during a training session to move the resistance valve from the first to the second configurations;
receive pressure measurement data from the pressure sensor; and,
determine at least one breathing performance indicator of the user based at least on a comparison of the target resistance level with the pressure measurement data from the pressure sensor.

27. An adaptor as claimed in claim 26, wherein the at least one breathing performance indicator of the user is additionally based on one or more of:
a number of times the pressure measurement data from the pressure sensor fell within or were greater than the target resistance level for more than a pre-defined amount of time, defined as successful attempts;
a number of times the actual pressure measurement data fell within or were greater than the target resistance level for less than a pre-defined amount of time, defined as unsuccessful attempts;
a number of times the actual pressure measurement data was less than the target resistance level, defined as unsuccessful attempts;
an amount of time taken to reach the threshold pressure required to move the resistance valve from the first to the second configurations;
an inhalation or exhalation time of a previous successful attempt;
a maximum air pressure generated, and/or an average air pressure generated over the training session;
an inhalation time of a previous inhalation and/or an average inhalation time over the training session;
an exhalation time of a previous exhalation and/or an average exhalation time;
an average resistance level achieved during the training session;
a total measurement time, which defines the length of a training session; and,
an average pressure time product achieved and/or a total pressure time product achieved during the training session, wherein:
an attempt is recorded as successful if the user 1) generates an air pressure falling within or above their target resistance level, and 2) remains within or above their target resistance level for a minimum amount of time.

28. An adaptor as claimed in claim 26, wherein the software application is further configured to display the pressure measurement data in real-time during the training session; and wherein the software application is additionally configured to update and display in real-time the at least one breathing performance indicator of the user as a training session progresses.

29. An adaptor as claimed in claim 26, wherein the software application is configured to determine at least two breathing performance indicators; and wherein two or more of the at least two breathing performance indicators are combined to form at least one breathing performance indicator which is more user-friendly; and wherein the software application is additionally configured to update and display the at least one user-friendly breathing performance indicator of the user in real-time during the training session.

30. An adaptor as claimed in claim 26, wherein the software application is configured to analyse the pressure measurement data and/or the at least one breathing performance indicator, and provide the user with one or more automated suggestions on how to improve their usage of the adaptor and optimise their training.

31. A method according to claim 19, further comprising:
at least partially locating a selectively removable plug within the first end of the intelligent adaptor to substantially occlude the passageway of the adaptor such that in use, a substantially sealed chamber is created between at least the user's lungs and the plug, thereby enabling the user to generate a pressure within the passageway upon activation of their respiratory muscles under isometric conditions to enable performance of an isometric respiratory muscle strength test.

32. A method according to claim 31, wherein the resistance level and an associated training plan is additionally determined based on user input regarding at least their previous and current lung health, including activity levels and smoking habits, any previous breathing training sessions, and future goals.
